(19) United States
(12) Patent Application Publication
(10) Pub. No.: US 2007/0231405 A1
(43) Pub. Date: Oct. 4, 2007

(54) Compositions and Methods Using Same for Treating Neurodegenerative Disorders

(75) Inventor: Meir Gorban, Rome (IT)

Correspondence Address: Martin D. Moynihan;PRTSI, P.O.Box 16446, Arlington, VA, 22215, US

(73) Assignee: FUTURE PRODUCTS MANAGEMENT S.A., WICKHAMS CAY I (VG)

(21) Appl. No.: 11/628,216

(22) PCT Filed: Jun. 1, 2005
(86) PCT No.: PCT/IL05/00573

§ 371 Date: Dec. 1, 2006

(30) Foreign Application Priority Data
Jun. 1, 2004 (IL) 162288

(51) Int. Cl. A61K 36/16; A61K 36/258; A61K 36/38; A61K 36/8962; A61P 27/06

(52) U.S. Cl. 424/601; 424/630; 424/639; 424/641; 424/646; 424/655; 424/667; 424/682; 424/702; 424/728; 424/730; 424/737; 424/752

(57) ABSTRACT

Compositions and methods of using same are provided for the treatment of neurodegenerative disorders such as Glaucoma, Multiple Sclerosis, Myasthenia Gravis, Diabetic Neuropathy, Cerebrovascular accident, spinal cord injuries, ALS, Parkinson's disease and Idiopathic dementia.

### FIELD AND BACKGROUND OF THE INVENTION

The present invention relates to novel compositions and methods of using same which can be used for treating neurodegenerative disorders such as glaucoma, multiple sclerosis, myasthenia gravis, diabetic neuropathy, cerebrovascular accident, spinal cord injuries, ALS, Parkinson's disease and Idiopathic dementia.

It is commonly accepted that components of the Central Nervous System (CNS), including the brain, spinal cord, olfactory, optic nerve and retina of the eye are unable to recover following damage of whatever cause and the morphological and functional damage engendered by the disease or injury is usually permanent. Medication is available to provide symptomatic relief only, although in some cases it is also able to slow down the course of the disease.

An example of an incurable neurodegenerative disease is glaucatomus neuropathy (Glaucoma). Glaucoma is a major public health problem affecting about sixty six million people around the world with approximately three million patients in the U.S.A. alone. Glaucoma is an optic nerve degenerative disease that causes loss of vision and blindness. A characteristic feature of glaucoma is the progressive death of retinal ganglion cells. In many cases this is caused by an increased intraocular pressure, which leads to axonal degeneration in the optic nerve and loss of ganglion cells.

Early diagnosis is very important in the treatment of glaucoma. Although there is no cure for this disease, it can be controlled to some extent with surgery and medication. Prolonged usage of drugs is required. Some medicines are known to cause headaches or produce other side effects. There is thus an urgent need for new medicines that specifically arrest ganglion cell death.

Parkinson's disease is another common incurable neurodegenerative disease found in the elderly population. About 50,000 new cases of this disease are reported in USA alone. Major clinical manifestations of this disease are bradekinesia (difficulty in voluntary movements), rigidity, body tremors, postural instability and impaired balance. This disease is marked by a loss of pigmented neurons in the substantia nigra in the mid brain region. Dopaminergic neurons in the substantia nigra and other catecholamine neurons in the brainstem are selectively lost in this condition. The cause of cell death or impairment is unknown.

Parkinson's disease cannot be cured at present. Medication is only available to provide relief from the symptoms. There are two general approaches to the treatment of Parkinson's disease with medication. The first approach attempts to retard the loss of dopamine in the brain and the second approach attempts to treat the symptoms of Parkinson's disease by other means. Dopamine agonists carry a high risk of short-term side effects such as nausea, vomiting, dizziness, light-headedness, confusion, and hallucinations. Anticholinergics are used to restore the balance between the two brain neurotransmitters dopamine and acetylcholine, by reducing the amount of acetylcholine. This reduces tremor and muscle stiffness in patients with Parkinson's. These medications, however, can impair memory and thinking, especially in older people; therefore, they are rarely used today.

Levadopa is the most widely used drug for Parkinson's disease. It delays the onset of the more debilitating symptoms for some time. It does not however, alleviate all symptoms. Levadopa is not without side effects. Nausea, vomiting, low blood pressure, involuntary movements and restlessness have been observed in patients using this medication.

As described above, diseases and conditions of the brain are generally incurable. All available treatments are symptomatic and relief is limited. Prolonged usage of drugs has a variety of adverse effects on the patients. Treatment often calls for surgical intervention. Invasive procedures are not risk free since they have the potential to cause irreversible damage. U.S. Pat. No. 6,405,079 while discussing the various shortfalls in treating neurological conditions teaches an invasive procedure that requires permanent implantation of electrodes. U.S. Pat. No. 6,277,372 also suggests that there is a general lack of treatment for neurodegenerative diseases. It traces these diseases to defects in neural circuitry and offers a method of treating neurodegenerative diseases by transplanting porcine neural cells into a human subject. Such a method requires extremely advanced medical procedures and can be very expensive. Additionally, as pointed out in U.S. Pat. No. 6,277,372, such treatment is usually accompanied by administration of immunosuppressive drugs. Moreover, implantation of foreign neural matter as an accepted and safe course of treatment is debatable.

The use of nutritional compounds (including vitamins, minerals, medicinal herbs and other compounds) has been suggested in U.S. Pat. App. No. 20040001896 and a number of compositions are commercially available such as e.g. Brain Sustain™, Neuroplus, and Senescegarl™. However, the exact components of the composition and concentration of each component as well as the administration of the composition in a particular treatment regimen can profoundly affect the therapeutic potency of the composition.

It is therefore the object of this invention to overcome the drawbacks described in the currently available treatments and provide a safe and natural composition for the treatment of conditions affecting the neurological system.

### SUMMARY OF THE INVENTION

It is an object of the present invention to provide methods, uses thereof and pharmaceutical compositions for treating neurodegenerative diseases.

It is another object of the present invention to provide methods and pharmaceutical compositions for treating glaucatomus neuropathy.

Hence, according to one aspect of the present invention there is provided a pharmaceutical composition comprising as active ingredients Vitamin C, Vitamin E, Gingko biloba and at least one substance selected from the group consisting of a vitamin, a mineral, an amino acid and a herb and a physiologically acceptable pharmaceutical carrier or diluent.

According to another aspect of the present invention, there is provided a use of the pharmaceutical composition for treating a neurodegenerative disorder.

According to yet another aspect of the present invention, there is provided a use of the pharmaceutical composition for the manufacture of a medicament identified for treating a neurodegenerative disorder.

According to yet another aspect of the present invention, there is provided a method of treating a neurodegenerative disorder in a subject, the method comprising providing to a subject in need thereof the pharmaceutical composition, thereby treating the neurodegenerative disorder in the subject.

According to further features in preferred embodiments of the invention described below, the vitamin is selected from the group consisting of Vitamin A, Vitamin B1, Vitamin B2, Vitamin B3, Vitamin B5, Vitamin B6, Vitamin B12, Vitamin D, Vitamin H, Choline, Folic acid and Paraamino Benzoic Acid (PABA).

According to still further features in preferred embodiments of the invention described below, the mineral is selected from the group consisting of Calcium, Chromium, Copper, Iodine, Iron, Magnesium, Manganese, Phosphorus, Potassium, Selenium (Picolinate) and Zinc.

According to yet further features in preferred embodiments of the invention described below the herb is selected from the group consisting of Allum Sativum (garlic), Black Currant (Ribes nigra), Bromlain, Echinacea, Ginseng (panax), Ginseng (Siberian), Hydrastasis, Medicago sativa (Alfalfa), Passiflora, Ruscus aculeatus, St. John wort (Hypericum perforatum) and Vaccinium myrtillus.

According to further features in preferred embodiments of the invention described below, the as least one substance is selected from the group consisting of DMAE (Dimethylaminoethanol), Flavonoids (Rutin), Glutathione, Inisitol, Lycopene, Melatonin, Omega 3 fatty acids, Phosphatidyl choline (Lecithine), Phosphatidyl serine, Quercetine and Ubiquitine (Q10).

According to further features in preferred embodiments of the invention described below, the neurodegenerative disorder is selected from the group consisting of Parkinson's disease, multiple sclerosis, amyotrophic lateral sclerosis, myasthenia gravis, diabetic neuropathy, cerebrovascular accident, spinal cord injuries, glaucatomus neuropathy, autoimmune encephalomyelitis, Alzheimer's disease, idiopathic dementia and Huntington's disease.

According to yet another aspect of the present invention, there is provided a pharmaceutical composition comprising as active ingredients Vitamin C, Vitamin E, Gingko biloba and at least one substance selected from the group consisting of Zinc, Selenium picolinate, Vitamin A, Vitamin B₁, Vitamin B₂, Vitamin B₃, Vitamin B₅, Vitamin H, folic acid, Chromium, Manganese, Flavonoid, Phosphatidyl choline, Dimethylaminoethaonol, Omega 3 fatty acid, Arginine, Vaccinium myrtillus extract and Paraamino benzoic acid (PABA) and a physiologically acceptable pharmaceutical carrier or diluent.

According to yet another aspect of the present invention there is provided a use of the pharmaceutical composition for treating glaucatomus neuropathy.

According to yet another aspect of the present invention there is provided a use of the pharmaceutical composition for the manufacture of a medicament identified for treating glaucatomus neuropathy.

According to yet another aspect of the present invention there is provided a method of treating glaucatomus neuropathy in a subject, the method comprising administering to a subject in need thereof the pharmaceutical composition, thereby treating the glaucatomus neuropathy in the subject.

According to further features in preferred embodiments of the invention described below, the pharmaceutical composition further comprises one or more substances selected from the group consisting of Vitamin B₆, Vitamin B₁₂, Vitamin D, choline, folic acid, Calcium, Bromlain, Copper, Iodine, Magnesium, Phosphorus, Potassium, Phosphatidyl serine, Quercetine, Ubiquitine, Glutathione, Inisitol, melatonin, Aspartate, Cysteine, Glutamate, Glutamine, Glycine, Histidine, Isoleucine, Leucine, Lysine, Methionine, Phenylalanine, Proline, Serine, Threonine, Tryptophan, Tyrosine, Valine, Allum Sativum (garlic) Black Currant (Ribes nigra), Echinacea, Ginseng (panax), Ginseng (Siberian), Hydrastasis, Medicago sativa (Alfalfa), Passiflora, Ruscus aculeatus and St. John wort (Hypericum perforatum).

According to further features in preferred embodiments of the invention described below, the substance is selected from the group consisting of Potassium, Magnesium, Vitamin B₆, Vitamin B₁₂ and phosphatidyl serine.

According to further features in preferred embodiments of the invention described below, Vitamin C, Vitamin E, Gingko biloba and substances are each in individual unit dosage forms.

According to further features in preferred embodiments of the invention described below, the composition is formulated in a unit dosage form.

According to further features in preferred embodiments of the invention described below, the unit dosage form is formulated for oral and/or rectal administration.

According to further features in preferred embodiments of the invention described below, the unit dosage form is selected from the group consisting of pills, tablets, capsules, gel-capsules and suppositories.

According to further features in preferred embodiments of the invention described below, the unit dosage form comprises 500-6000 mg of Vitamin C, 200-8000 IU of Vitamin E and 40-160 mg of Gingko biloba extract.

The present invention successfully addresses the shortcomings of the presently known configurations by providing novel compositions for the treatment of neurodegenerative diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-C are computerized images illustrating the recovery of the optic nerve of the right eye of patient 1 diagnosed with glaucatomus neuropathy as represented in his visual field from 31 Jan. 2002 to 30 Jun. 2002 following treatment with the composition as detailed in table 2 below, administered directly following the initial field of vision analysis. [The darkened areas of the diagram are the parts which are not seen by the patient. The midline dark area on the left of the midline represents the normal blind spot]. FIG. 1A is a computerized image from a field of vision analysis performed on 31 Jan. 2002 immediately prior to treatment. FIG. 1B is a computerized image from a field of vision analysis performed on 25 Mar. 2002. FIG. 1C is a computerized image from a field of vision analysis performed on 30 Jun. 2002.

FIGS. 2A-B are computerized images illustrating the recovery of the optic nerve of the right eye of patient 2 diagnosed with glaucatomus neuropathy as represented in his visual field from 18 Apr. 2002 to 25 Sep. 2002 following treatment with the composition as detailed in table 2 below, administered directly following the initial field of vision analysis. FIG. 2A is a computerized image from a field of vision analysis performed on 18 Apr. 2002 immediately prior to treatment. FIG. 2B is a computerized image from a field of vision analysis performed on 25 Sep. 2002.

FIGS. 3A-J are computerized images illustrating the recovery of the optic nerve of both eyes of patient 3 diagnosed with glaucatomus neuropathy following treatment with the composition as detailed in table 2 below, administered directly following the initial field of vision analysis. FIG. 3A is a computerized image from a field of vision analysis on the right eye performed on 21 Jun. 1999 immediately prior to treatment. FIG. 3B is a computerized image from a field of vision analysis performed on the right eye on 4 Jul. 1999. FIG. 3C is a computerized image from a field of vision analysis performed on the right eye on 2 Dec. 1999. FIG. 3D is a computerized image from a field of vision analysis performed on the right eye on 9 Mar. 2000. FIG. 3E is a computerized image from a field of vision analysis performed on the right eye on 24 May 2001. FIG. 3F is a computerized image from a field of vision analysis on the left eye performed on 21 Jun. 1999 immediately prior to treatment. FIG. 3G is a computerized image from a field of vision analysis performed on the left eye on 4 Jul. 1999. FIG. 3H is a computerized image from a field of vision analysis performed on the left eye on 2 Dec. 1999. FIG. 3I is a computerized image from a field of vision analysis performed on the left eye on 9 Mar. 2000. FIG. 3J is a computerized image from a field of vision analysis performed on the left eye on 24 May 2001.

### DESCRIPTION OF THE PREFERRED EMBODIMENTS

The present invention relates to pharmaceutical compositions and methods of using same for the treatment of neurodegenerative disorders, such as glaucatomus neuropathy.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Neurodegenerative disorders, which are characterized by loss of neuronal functions, such as Parkinson's disease and glaucatomus neuropathy, cannot be efficiently treated using conventional drug therapy since such drugs have no effect on the underlying disease process which is typically caused by neuronal degeneration. Consequently, drug therapy cannot fully compensate for the increasing loss of neuronal cells.

While reducing the present invention to practice, the present inventors uncovered novel compositions that can be used to treat neurodegenerative disorders.

Compositions of the present invention are provided in concentrations that are preferably higher than ordinarily needed for normal physiology. The compositions administered comprise components that are either reduced in the patient due to the disease or general condition or are required for inducing recovery.

As is illustrated herein below and in the Examples section that follows, administration of the compositions of the present invention to Glaucoma patients resulted in an unprecedented recovery where the visual field showed definitive improvement. This improvement testifies to the recovery of the retina and its connections to the brain. These results are considered virtually impossible in Glaucoma patients and place the compositions of the present invention as leading therapeutics for the treatment of neurodegenerative disorders such as Glaucoma.

Thus, according to one aspect of the present invention there is provided a pharmaceutical composition comprising as active ingredients Vitamin C, Vitamin E, Gingko biloba and at least one substance selected from the group consisting of a vitamin, a mineral, an amino acid and a herb and a physiologically acceptable pharmaceutical carrier or diluent for the treatment of a neurodegenerative disorder.

As used herein, the phrase “neurodegenerative disorder” refers to any disorder, disease or condition of the nervous system (preferably CNS) which is characterized by gradual and progressive loss of neural tissue, neurotransmitter, or neural functions. Examples of neurodegenerative disorders include, but are not limited to, Parkinson's disease, glaucatomus neuropathy, multiple sclerosis, myasthenia gravis, amyotrophic lateral sclerosis, autoimmune encephalomyelitis, diabetic neuropathy, cerebrovascular accident (stroke), Alzheimer's disease, idiopathic dementia and Huntington's disease. The phrase “neurodegenerative disorder” also refers to neural trauma e.g., injuries such as spinal cord injuries and head injuries.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to a vitamin, a mineral, an amino acid, a compound or a herb accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

As used herein “Vitamin C” refers to ascorbic acid, which is an essential nutrient found in fruit and vegetables. It comprises a range of biological functions. It is required for the production and maintenance of collagen as well as for the metabolism of folic acid, tyrosine and tryptophan. It is also known to enhance the immune response thereby protecting against infection, and it is important in the production of thyroxine. Of importance, Vitamin C contains antioxidant properties. Vitamin C of the present invention refers to a synthetic or natural form of Vitamin C, such as the Vitamin C synthesized extracted from corn syrup or sago palm. Alternatively, Vitamin C may be extracted from other natural sources such as for example rose hips, acerola cherries, peppers, or citrus fruits. Vitamin C of the present invention also refers to mineral ascorbates (such as sodium, potassium, calcium, zinc, molybdenum, chromium and manganese ascorbates), ascorbyl palmitate and D-isoascorbic acid. Vitamin C is commercially available from such Companies as Herbalife, Doctors Trust and Natrol.

As used herein, the term “Vitamin E” refers to any one or combination of the eight forms of Vitamin E comprising the four tocopherols (αβγδ) and the four tocotrienols (αβγδ) including the succinate, nicotinate and acetate salts derivatives thereof. In addition, each of these compounds has a “d” form, which is the natural form, and a “d1” form, which is the synthetic form. Preferably, the composition of the present invention comprises d-α tocopherol or a salt derivative thereof as this is the most active form of Vitamin E. Vitamin E may be extracted from such foods as vegetable oils (olive, sunflower, and safflower oils), nuts, whole grains, and green leafy vegetables. Vitamin E is known to play an important role in the body as an antioxidant. Vitamin E is commercially available from such Companies as Herbalife or Doctors Trust.

As used herein the term “Gingko biloba” refers to the active ingredients extracted from the gingko biloba tree including Ginkgoflavoneglycos, Bilobalide, and terpenelactones including ginkgolides A, B and C or plant portions thereof. Gingko biloba is a powerful antioxidant as well as a known vasodilator.

Extracts of the gingko biloba tree are commercially available, with varying concentrations of the active ingredients. One example of a standardized extract is EGb761 (Natures Way, U.S.A.) comprising approximately 24% flavone glycosides (primarily quercetin, kaempferol and isorhamnetin) and 6% terpene lactones (2.8-3.4% ginkgolides A, B and C, and 2.6-3.2% bilobalide). Ginkgolide B and bilobalide account for about 0.8% and 3% of the total extract, respectively. Other constituents include proanthocyanadins, glucose, rhamnose, organic acids, D-glucaric and ginkgolic acids.

Other examples of standardized gingko biloba extracts include, but are not limited to the three formulations which are available from Linnea (Switzerland) (EPG 246: 24% Gingko flavonglycosides, 6% Terpene lactones (as used in the Examples section below); G 328: 32% Gingko flavonglycosides, 8% Terpene lactones; G 320: 32% Gingko flavonglycosides, without Terpene lactones).

The active ingredients may be synthesized chemically or extracted from their natural source. Initially, the leaves are dried and milled, following which they are pulverized and mixed with organic solvents, which liberate the chemical components of the leaves. The process is repeated a number of times to ensure purity. The crude extract is then further refined to a point where the flavonoids make up a precise concentration of the mixture.

Dependent on the intended use, the concentration of Vitamin C in the pharmaceutical composition is between about 500-6000 mg and more preferably between about 1000-4000. The concentration of Vitamin E in the pharmaceutical composition is between about 200-8000 IU and more preferably between about 400-2000 IU and the concentration of Gingko biloba is between about 40-160 mg and more preferably between about 40-120 mg. As used herein in the specification and claims section that follows the term about means ±20%.

Specific examples of concentrations ranges preferably used for neurological disorders of interest are further provided hereinbelow.

Without being bound to any theory, it is believed that at least part of the therapeutic effect of the pharmaceutical composition of the present invention relies on the antioxidant properties of Vitamin C, Vitamin E and gingko biloba which are necessary for reducing the production of excess free radicals in the brain, a marker and common thread among such neurodegenerative disorders as Alzheimer's disease, Parkinson's disease, multiple sclerosis, and ALS. It is believed that the therapeutic effect of the pharmaceutical composition of the present invention may also stem from the vasodilatory properties of some of its components and also the connective tissue elasticity-enhancing properties of some of its components as well as their immuno-modulatory effects. The above mechanisms are not inclusive and many others may be in operation in effecting the neural recovery which this treatment modality provides.

As mentioned hereinabove, the pharmaceutical composition of this aspect of the present invention may comprise other vitamins, beyond the above described.

As used herein the term “vitamin” refers to a naturally occurring vitamin, a precursor, a salt derivative or a metabolite thereof, either in a natural or synthetic form which comprises therapeutic activity for the treatment of a neurodegenerative disorder.

Examples of vitamins which can be included in the pharmaceutical composition of this aspect of the present invention include, but are not limited to Vitamin A, Vitamin B₁, Vitamin B₂, Vitamin B₃, Vitamin B₅, Vitamin B₆, Vitamin B₁₂, Vitamin D, Vitamin H, Choline, Folic acid and Paraamino Benzoic Acid (PABA). The vitamins of the present invention are commercially available from such Companies as Herbalife, Doctors Trust and Natrol.

As mentioned above, the pharmaceutical composition of this aspect of the present invention may comprise a mineral.

As used herein, the term “mineral” refers to an element or chemical compound that is typically crystalline resulting from geological processes. A mineral of the present invention refers to an isolated mineral or a salt thereof. Examples of minerals that may be used in the present invention include but are not limited to Calcium, Chromium, Copper, Iodine, Iron, Magnesium, Manganese, Phosphorus, Potassium, Selenium (Picolinate) and Zinc.

The pharmaceutical composition of the present invention may also comprise a herb. As used herein, the term “herb” refers to a fresh or dried part of a plant or a whole plant or an extract thereof, which comprises therapeutic activity for the treatment of a neurodegenerative disorder. Examples of herbs that can be used in the present invention include, but are not limited to Allum Sativum (garlic), Black Currant (Ribes nigra), Bromlain, Echinacea, Ginseng (panax), Ginseng (Siberian), Hydrastasis, Medicago sativa (Alfalfa), Passiflora, Ruscus aculeatus, St. John wort (Hypericum perforatum) and Vaccinium myrtillus.

A wide range of methods are known in the art for the production of therapeutics from herbs. For example, herbs may be subjected to a polar (e.g., aqueous) extraction. The aqueous extract may then be filtered if necessary to remove large particles, and subsequently dried (e.g. by exposure to warm dry air (e.g., 65° C.) for a length of time such as three days to one week) to a powder. Alternatively, it is possible to use dry herbs directly by grinding to a powder.

The pharmaceutical composition of the present invention may also comprise a naturally occurring substance such as, for example, DMAE (Dimethylaminoethanol), Flavonoids (Rutin), Glutathione, Inositol, Lycopene, Melatonin, Omega 3 fatty acids, Phosphatidyl choline (Lecithine), Phosphatidyl serine, Quercetine and Ubiquitine (Q10). These substances comprise anti-neurodegenerative effects by means of their immuno-modulatory and anti-oxidising properties. All these substances may be bought commercially in any health food store.

Maximum suggested concentrations of the additional components are listed hereinbelow.

TABLE 1

| | Maximum suggested | |
|---|---|---|
| Component | concentrations | |
| Vitamin A | 25.000 | IU |
| Vitamin B1 | 150 | mg |
| Vitamin B2 | 150 | mg |
| Vitamin B3 | 150 | mg |
| Vitamin B5 | 500 | mg |
| Vitamin B6 | 180 | mg |
| Vitamin B12 | 400 | μg |
| Vitamin D | 2500 | IU |
| Vitamin H | 80 | mg |
| Choline | 200 | mg |
| Folic acid | 10 | mg |
| Paraamino Benzoic Acid (PABA) | 500 | mg |
| Calcium | 2000 | mg |
| Chromium | 300 | mg |
| Copper | 5 | mg |
| Iodine | 50 | mg |
| Magnesium | 1000 | mg |
| Manganese | 8 | mg |
| Phosphorus | 150 | mg |
| Iron | 75 | mg |
| Potassium | 6 | mg |
| Selenium | 500 | μg |
| Zinc | 40 | mg |
| Alanine | 700 | mg |
| Aspartate | 200 | mg |
| Arginine | 600 | mg |
| Cysteine | 150 | mg |
| Glutamate | 50 | mg |
| Glutamine | 50 | mg |
| Glycine | 200 | mg |
| Histidine | 120 | mg |
| Isoleucine | 100 | mg |
| Leucine | 150 | mg |
| Lysine | 100 | mg |
| Methinonine | 200 | mg |
| Phenylalanine | 700 | mg |
| Proline | 700 | mg |
| Serine | 300 | mg |
| threonine | 100 | mg |
| tryptophan | 300 | mg |
| tyrosine | 700 | mg |
| valine | 150 | mg |
| Folic acid | 100 | μg |
| DMAE | 150 | mg |
| Flavnoids | 200 | mg |
| Glutathione | 700 | mg |
| Inositol | 1000 | mg |
| lycopene | 20 | mg |
| Melatonin | 20 | mg |
| Omega 3 fatty acids | 10 | gm |
| Phosphatidyl choline | 1000 | mg |
| Phosphatidyl serine | 500 | mg |
| Quercetine | 1200 | mg |
| Ubiquitine | 5 | gm |
| Allum Sativum (garlic) | 700 | mg |
| Black Currant (Ribes nigra) | 200 | mg |
| Bromlain | 5000 | mg |
| Echinacea | 400 | mg |
| Ginseng (panax) | 300 | mg |
| Ginseng (Siberian) | 200 | mg |
| Hydrastasis | 200 | mg |
| Medicago sativa (Alfalfa) | 1000 | mg |
| Passiflora | 500 | mg |
| Ruscus aculeatus | 200 | mg |
| St. John wort (Hypericum perforatum) | 500 | mg |
| Vaccinium myrtillus | 500 | mg |

It is envisaged that the exact pharmaceutical composition is tailored for a particular subject's needs following blood and urine sample analysis.

Tailoring is effected according to the subject's neurodegenerative disorder, severity of disorder, age, weight and sex as well as the length of treatment course.

The present inventors have shown through laborious experimentation that the pharmaceutical composition may be tailored for the neurodegenerative disorder Glaucatomus neuropathy.

Thus, according to another aspect of the present invention there is provided a pharmaceutical composition for treating Glaucatomus neuropathy.

Glaucatomus neuropathy is a neurodegerative disorder in which patients suffer a progressive loss of their visual fields as result of progressive atrophy of their optic nerve fibers which is followed by death of the ganglion cells in the retina. Present-day treatment of glaucoma involves lowering intraocular pressure thereby arresting the process of optic nerve atrophy. Lowering the intraocular pressure, however, does not lead to the recovery of the optic nerve neurons in the retina and the optic nerve.

The pharmaceutical composition of this aspect of the present invention comprises Vitamin C, Vitamin E, Gingko biloba and at least one substance selected from the group consisting of Zinc, Selenium picolinate, Vitamin A, Vitamin B₁, Vitamin B₂, Vitamin B₃, Vitamin B₅, Vitamin H, folic acid, Chromium, Manganese, Flavonoid, Phosphatidyl choline, Dimethylaminoethaonol, Omega 3 fatty acid, Arginine, Vaccinium myrtillus extract and Paraamino benzoic acid (PABA). Preferably the substances are Zinc, Selenium picolinate, Vitamin A, Vitamin B₁, Vitamin B₂, Vitamin B₃, Vitamin B₅, folic acid and Paraamino benzoic acid (PABA).

The pharmaceutical composition of this aspect of the present invention may further comprise one or more substances selected from the group consisting of Vitamin B₆, Vitamin B₁₂, Vitamin D, choline, folic acid, Calcium, Bromlain, Copper, Iodine, Magnesium, Phosphorus, Potassium, Phosphatidyl serine, Quercetine, Ubiquitine, Glutathione, Inisitol, melatonin, Aspartate, Cysteine, Glutamate, Glutamine, Glycine, Histidine, Isoleucine, Leucine, Lysine, Methionine, Phenylalanine, Proline, Serine, Threonine, Tryptophan, Tyrosine, Valine, Allum Sativum (garlic) Black Currant (Ribes nigra), Echinacea, Ginseng (panax), Ginseng (Siberian), Hydrastasis, Medicago sativa (Alfalfa), Passiflora, Ruscus aculeatus and St. John wort (Hypericum perforatum).

Preferably, the substances are Potassium, Magnesium, Vitamin B₆ Vitamin B₁₂ and phosphatidyl serine.

As mentioned above, the pharmaceutical composition of the present invention may be tailored according to the time after which the subject has begun a treatment course. As seen in the Examples section below, the pharmaceutical compositions of this aspect of the present invention are altered following three months and nine months of treatment. Typical pharmaceutical compositions and concentrations of the components thereof for the treatment of glaucatomus neuropathy are listed in Table 2 of the Examples section herein below.

As mentioned above, the pharmaceutical composition of the present invention may be tailored according to a particular neurodegenerative disorder.

For example a pharmaceutical composition for the treatment of Multiple Sclerosis preferably comprises selenium (e.g., 200-400 μg), bromlain (e.g., 250-3500 mg) and flavnoids (e.g., 100-150 mg) as well as the three active agents—Vitamin E (200-8000 I.U.), Vitamin C (e.g., 500-6000 mg) and Gingko biloba (40-160 mg).

A pharmaceutical composition for the treatment of Myasthenia Gravis preferably comprises Selenium (e.g., 200-400 μg), Vitamin B6 (e.g., 30-180 mg), Vitamin A (e.g., 5000-25,000 IU) and Ginseng Panax (e.g., 100-200 mg) as well as the three active agents—Vitamin E (200-8000 I.U.), Vitamin C (e.g., 500-6000 mg) and Gingko biloba (40-160 mg).

A pharmaceutical composition for the treatment of Diabetic Neuropathy preferably comprises Selenium (e.g., 200-400 μg), Vitamin B₆ (e.g., 30-180 mg), and Bromlain (e.g., 250-3500 mg) as well as the three active agents—Vitamin E (200-8000 I.U.), Vitamin C (e.g., 500-6000 mg) and Gingko biloba (40-160 mg).

A pharmaceutical composition for the treatment of Cerebrovascular accident (stroke) and spinal cord injuries preferably comprises Bromlain (e.g., 250-3500 mg) as well as the three active agents—Vitamin E (200-8000 I.U.), Vitamin C (e.g., 500-6000 mg) and Gingko biloba (40-160 mg).

A pharmaceutical composition for the treatment of Amyotrophic Lateral Sclerosis (ALS) preferably comprises Bromlain (e.g., 250-3500 mg) and Selenium (e.g., 200-400 μg), as well as the three active agents—Vitamin E (200-8000 I.U.), Vitamin C (e.g., 500-6000 mg) and Gingko biloba (40-160 mg).

A pharmaceutical composition for the treatment of Parkinson's disease preferably comprises Ginseng Panax (e.g., 100-200 mg) as well as the three active agents—Vitamin E (200-8000 I.U.), Vitamin C (e.g., 500-6000 mg) and Gingko biloba (40-160 mg).

A pharmaceutical composition for the treatment of Idiopathic Dementia preferably comprises Vitamin B₁ (e.g., 10-150 mg) and Vitamin B₃ (e.g., 20-150 mg) as well as the three active agents—Vitamin E (200-8000 I.U.), Vitamin C (e.g., 500-6000 mg) and Gingko biloba (40-160 mg).

Preferably, the compositions of the present invention also comprise minerals such as Magnesium (e.g., 30-1000 mg), Potassium (e.g., 2-4 mg) and Zinc (e.g., 15-40 mg).

The components of the pharmaceutical composition may each be formulated individually in unit dosage forms such that a subject is able to select the particular individual components and the quantities thereof to suit its particular needs. Alternatively some of the components may be formulated as one composition, so as to encourage patient compliance. For example, Vitamin E, Vitamin C and Gingko Biloba may be formulated in a single composition such as in a unit dosage form. Alternatively components specific to a particular neurodegenerative disorder may be formulated as one composition. Thus, for example a single composition may comprise Vitamin E, Vitamin C, Gingko Biloba, Selenium, Bromlain and Flavnoids and may be formulated in a unit dosage form for Multiple Sclerosis.

As mentioned, compositions of the present invention may be used for treating a neurodegenerative disorder in a subject in need thereof.

As used herein, the phrase “a subject in need thereof” refers to a mammal, preferably a human that suffers or is at a risk of suffering (i.e., pre-disposed such as genetically pre-disposed) from the diseases or conditions listed hereinabove. Examples of mammals other than human beings include domestic animals (e.g., cats, dogs, cattle, sheep, pigs, goats, poultry and equines).

As used herein in the specification and claims section that follows the terms “treatment” and “treating” mean alleviation of some or all of the symptoms associated with a disease, prolongation of life expectancy of patients having a disease, as well as complete recovery from a disease. The terms “treatment” and “treating” also mean prevention of a disease.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, intravenous, intranasal, or intraocular injections. Preferably, the pharmaceutical compositions of the present invention are administered orally.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

It will be further appreciated that the agents of the present invention may also be provided as food additives.

The phrase “food additive” [defined by the FDA in 21 C.F.R. 170.3(e)(1)] includes any liquid or solid material which is intended to be added to a food product. This material can, for example, include an agent having a distinct taste and/or flavor or a physiological effect (e.g., vitamins). The food additive composition of the present invention can be added to a variety of food products.

As used herein, the phrase “food product” describes a material consisting essentially of protein, carbohydrate and/or fat, which is used in the body of an organism to sustain growth, repair and vital processes and to furnish energy. Food products may also contain supplementary substances such as minerals, vitamins and condiments. See Merriani-Webster's Collegiate Dictionary, 10th Edition, 1993. The phrase “food product” as used herein further includes a beverage adapted for human or animal consumption.

A food product containing the food additive of the present invention can also include additional additives such as, for example, antioxidants, sweeteners, flavorings, colors, preservatives, nutritive additives such as vitamins and minerals, amino acids (i.e. essential amino acids), emulsifiers, pH control agents such as acidulants, hydrocolloids, antifoams and release agents, flour improving or strengthening agents, raising or leavening agents, gases and chelating agents, the utility and effects of which are well-known in the art.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several months or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as further detailed above.

It will be appreciated that the above-described therapeutic compositions of the present invention may be combined with other treatment modalities known in the art. For example, treating glaucatomus neuropathy using the compositions of the present invention can be combined with currently practiced medications, such as Propine™ Allergan Inc., as long as such combination therapy is free of adverse side effects.

Compositions of the present invention can be packed in a therapeutic or a nutritional kit.

For example, compositions of the present invention can be packaged in one or more containers with appropriate buffers and preservatives and used for directing therapeutic treatment.

Thus, active ingredients of the compositions of the present invention can be mixed in a single container or placed in individual containers. Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

The kit can also include instructions for determining if the tested subject is suffering from, or is at risk of developing a neurodegenerative diseases or disorder.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Examination of the Field of Peripheral Vision of Glaucoma Patients

Glaucoma-specific compositions generated according to the teachings of the present invention were administered to glaucoma patients and a therapeutic effect thereof was tested on their field of peripheral vision.

Experimental Procedures

Eligibility Criteria: The patients selected for this study met the following criteria: Patients had to be in good health as assessed by blood and urine examinations and to obtain a letter of permission from their physician. A psychological examination of the patients was performed to determine whether they would comply with the strict study regiment. Patients of any stage of the disease were selected for the study.

Patients were administered with the composition as detailed in Table 2 below. All components were administered individually in the form of capsules or pills.

TABLE 2

| | Months | | Months | | Months | |
|---|---|---|---|---|---|---|
| Substance | 1-3 | | 4-9 | | 10-12 | |
| Vitamin C | 1500 | mg | 2500 | mg | 2500 | mg |
| Vitamin E | 400 | I.U. | 800 | I.U. | 800 | I.U. |
| Gingko biloba | 40 | mg | 80 | mg | 80 | mg |
| (EPG 246: | | | | | | |
| 24% Ginkgo | | | | | | |
| flavonglycosides, | | | | | | |
| 6% Terpene | | | | | | |
| lactones) | | | | | | |
| Selenium | 200 | μg | 200 | μg | 200 | μg |
| Zinc | 25 | mg | 30 | mg | 30 | mg |
| Vitamin A | 10.000 | I.U. | 10.000 | I.U. | 10.000 | I.U. |
| Potassium | 4 | mg | 4 | mg | 4 | mg |
| Magnesium | 400 | mg | 400 | mg | 400 | mg |

| B1 | — | 50 | mg | 50 | mg |
|---|---|---|---|---|---|
| B2 | — | 50 | mg | 50 | mg |
| B3 | — | 50 | mg | 50 | mg |
| B5 | — | 50 | mg | 50 | mg |
| B6 | — | 80 | mg | 80 | mg |
| B12 | — | 80 | mcg | — | |
| Folic acid | — | 300 | μg | 300 | μg |
| PABA | — | 50 | mg | 50 | mg |

Examination of field of peripheral vision: A Computerized Automated Perimeter was used to test the field of vision, at three month intervals wherever possible. The patient was shown light targets of various size and brightness and the area of sight in the eye was noted. The data thus collected was analyzed and compared with data of normal population. In addition monthly blood and urine samples were analyzed.

Results

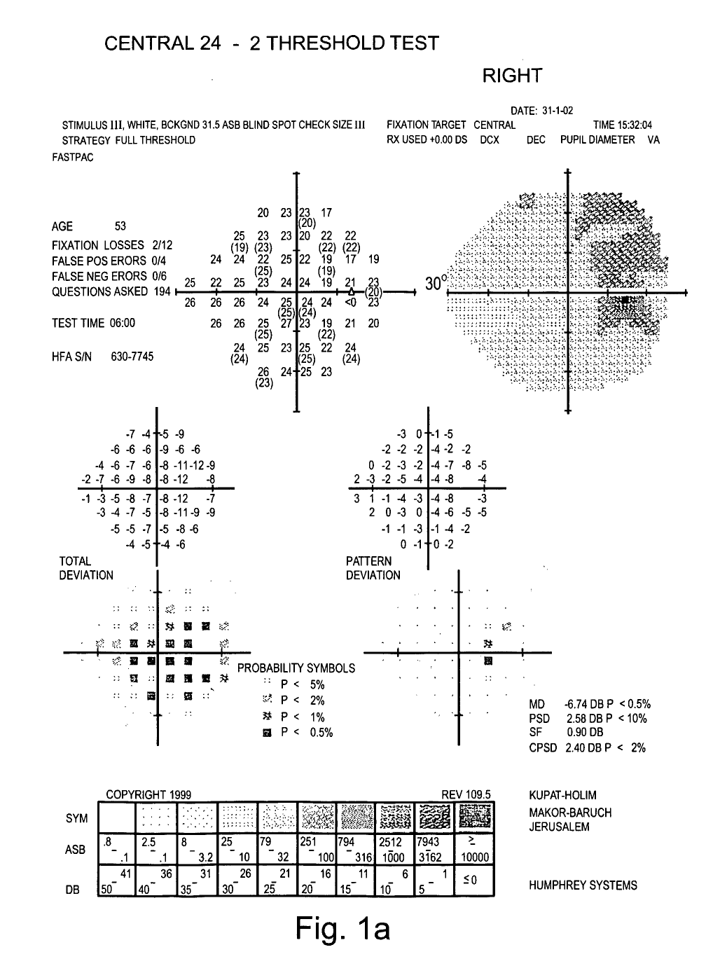

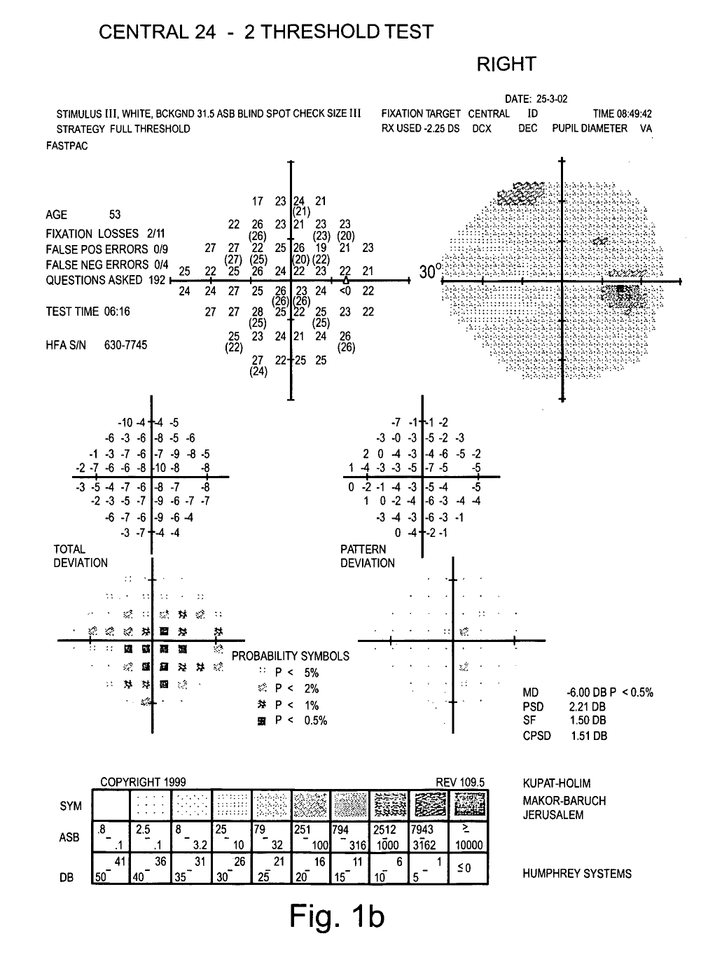

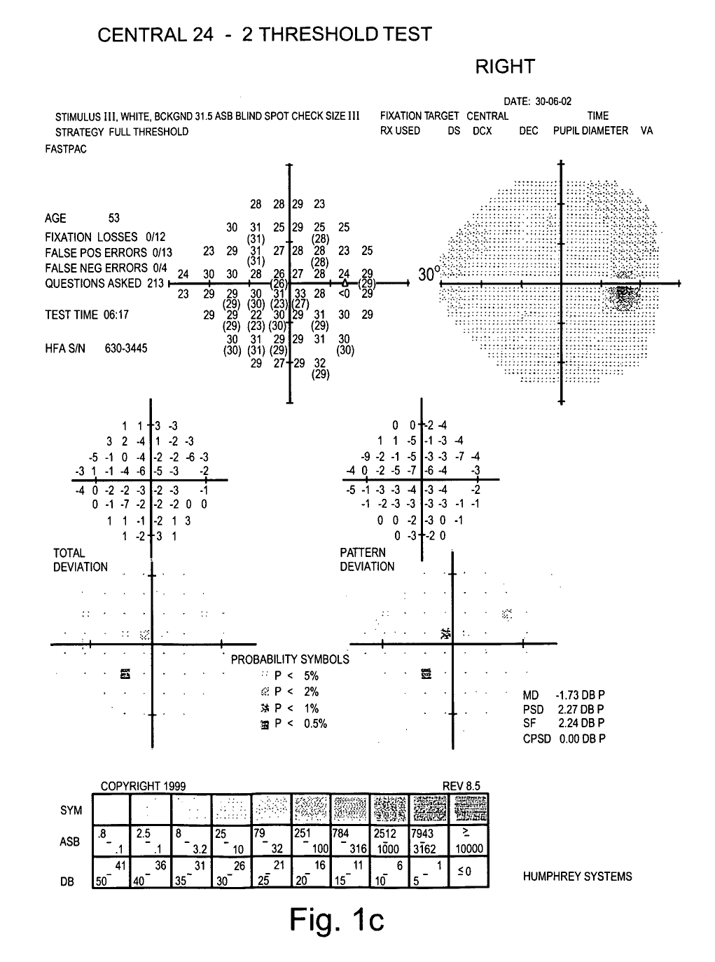

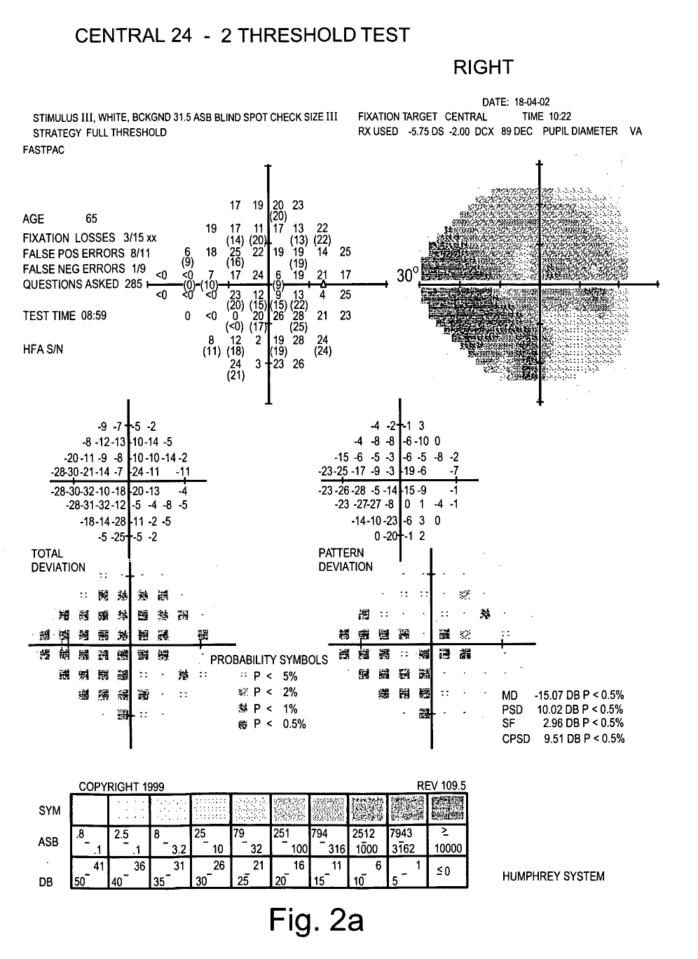

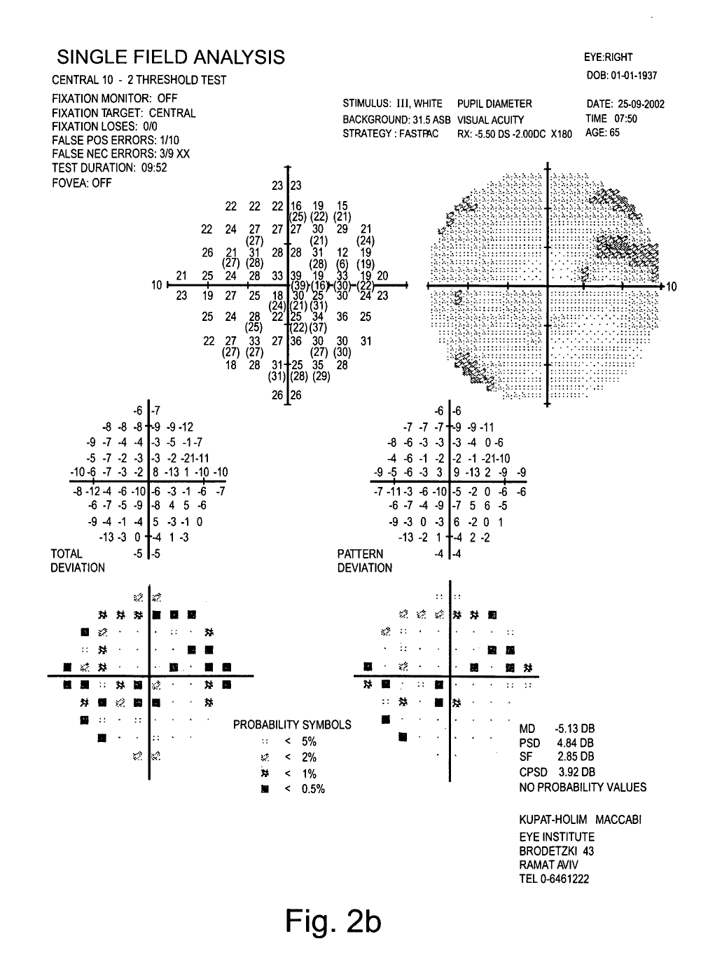

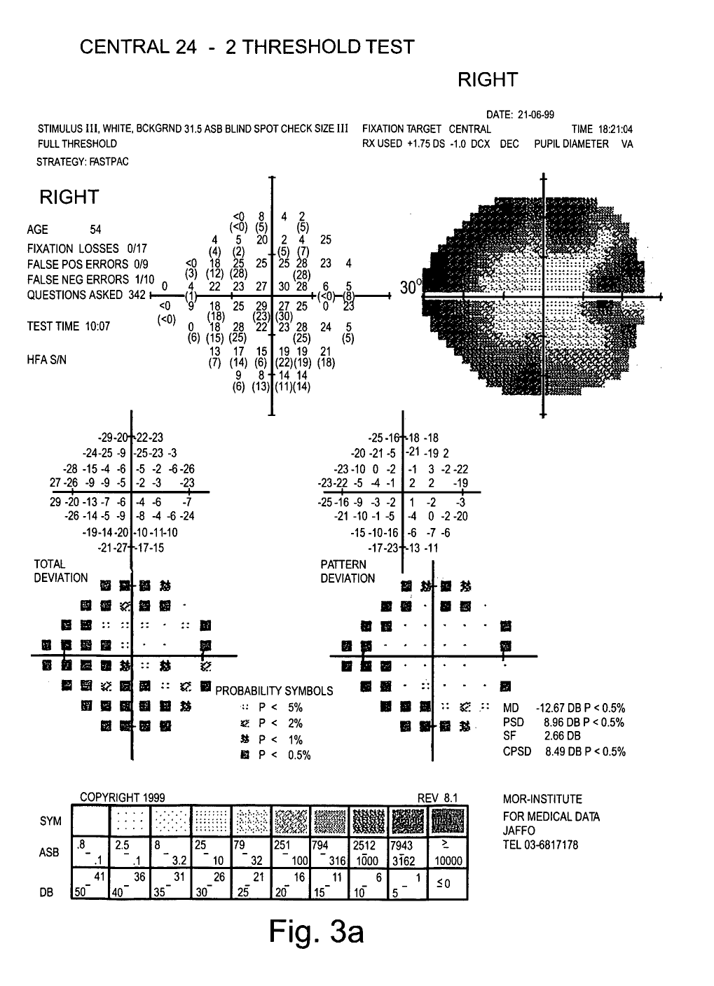

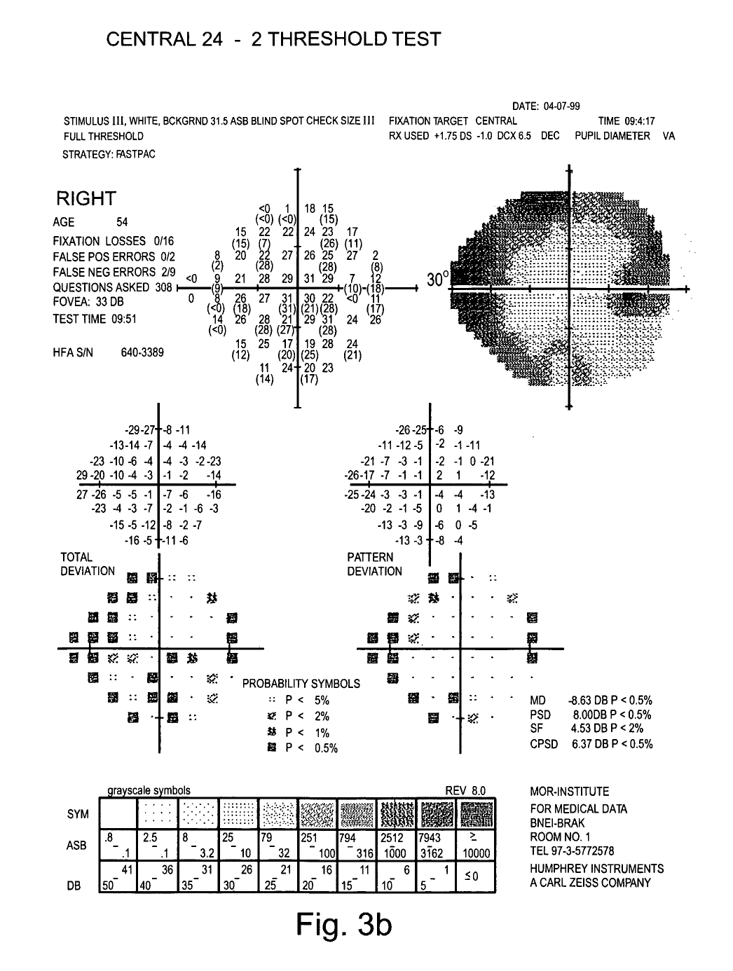

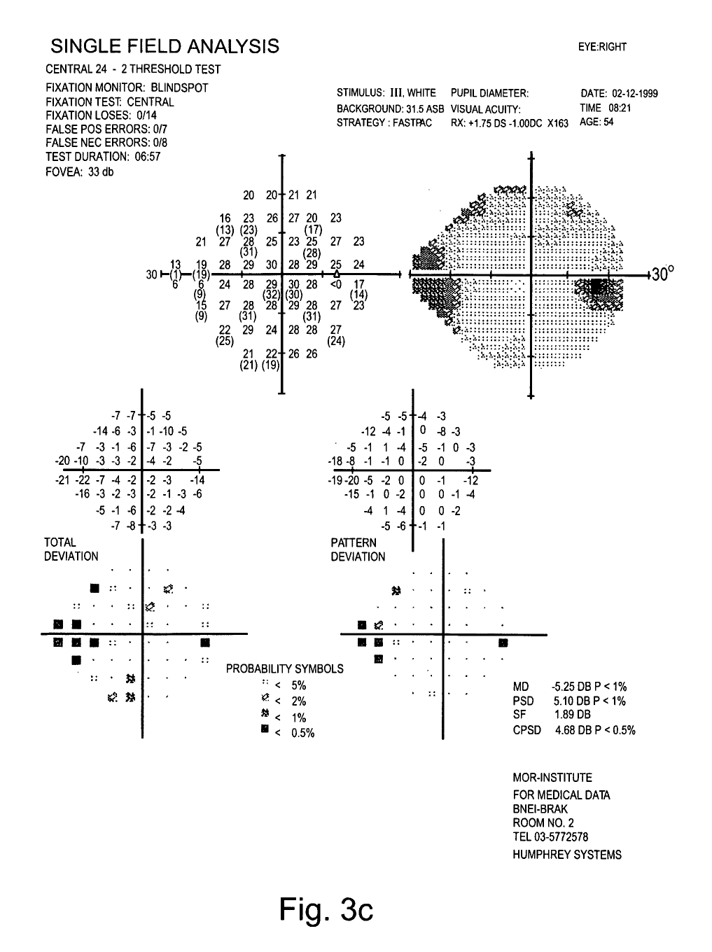

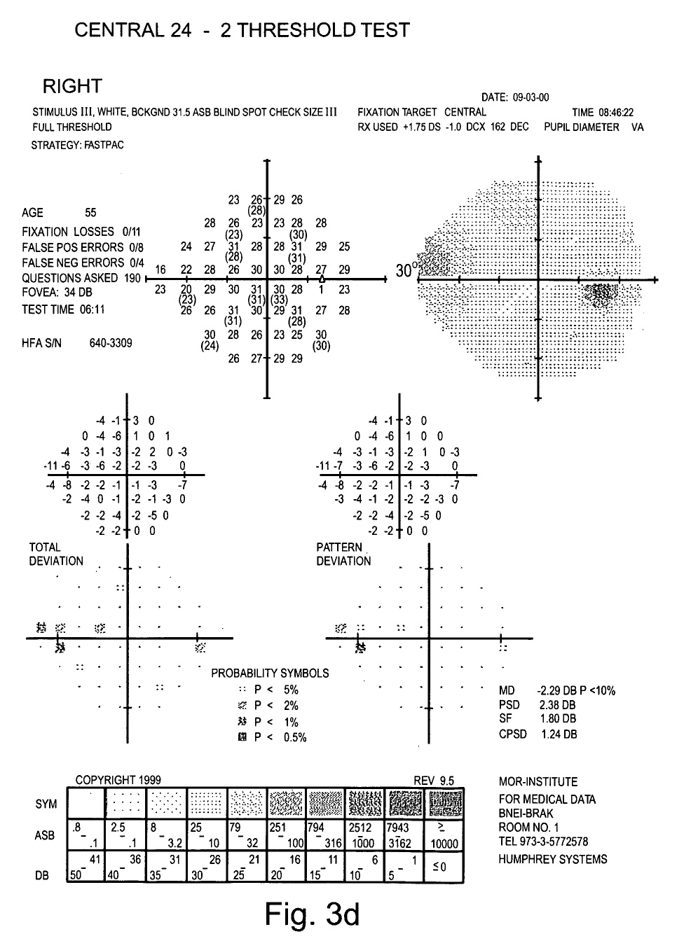

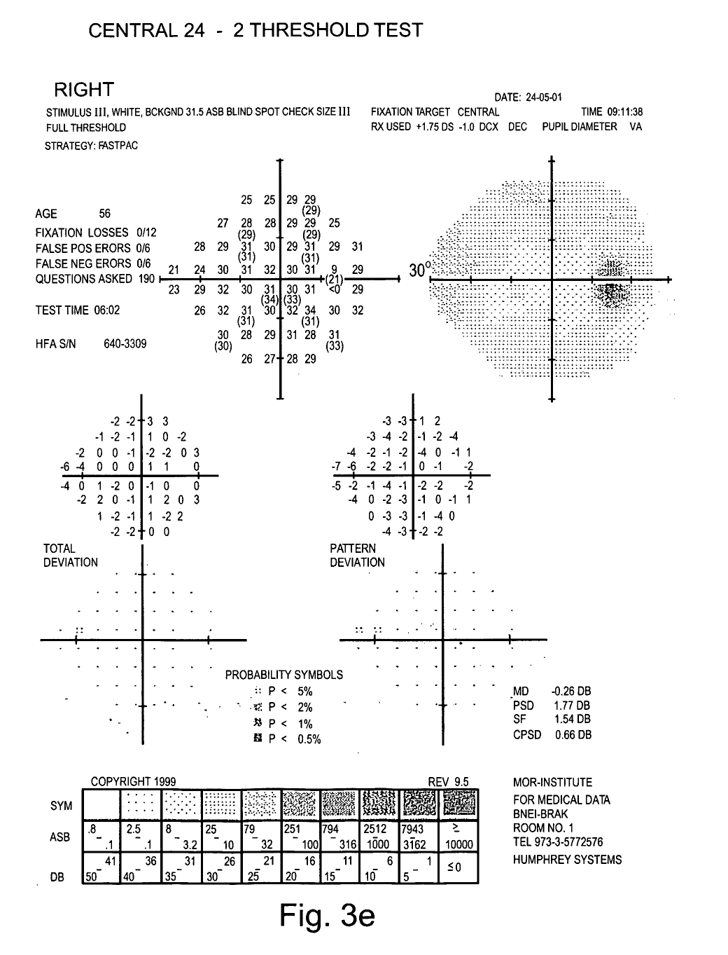

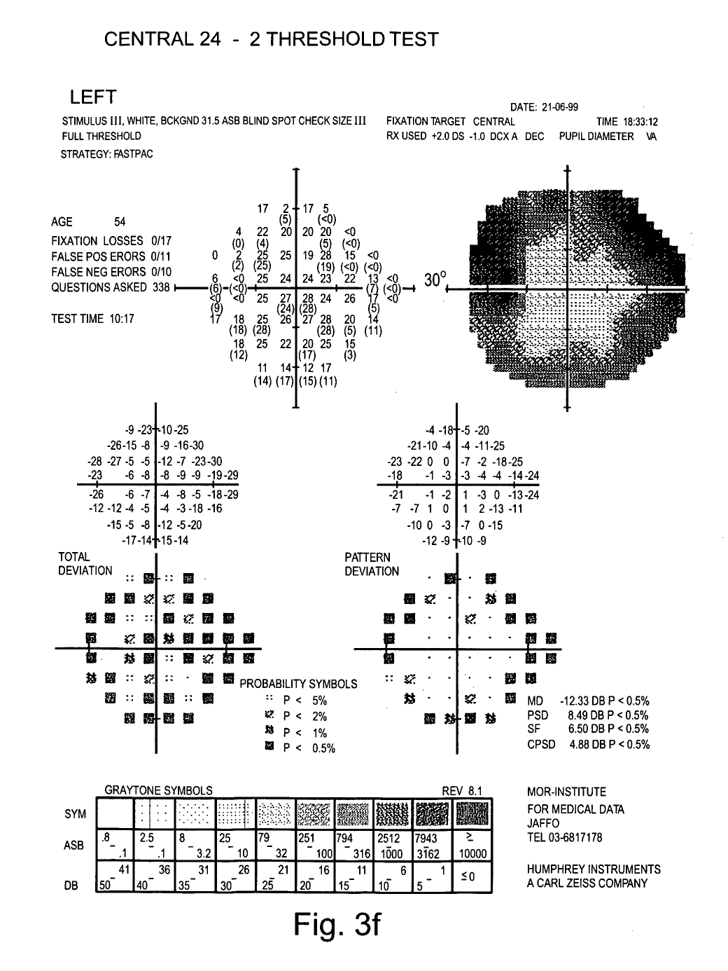

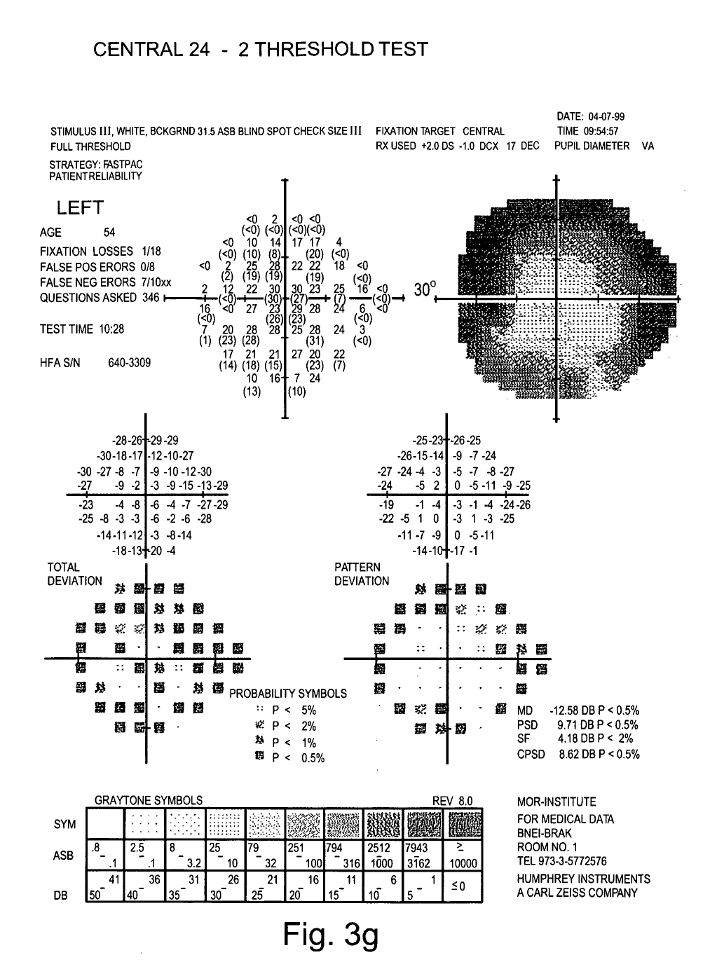

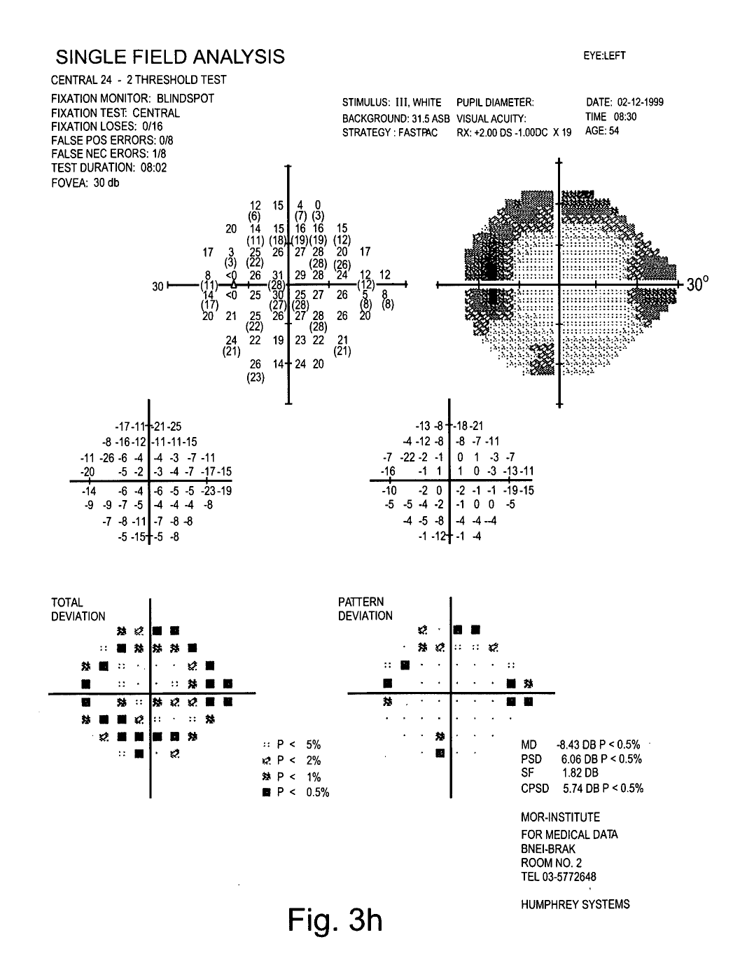

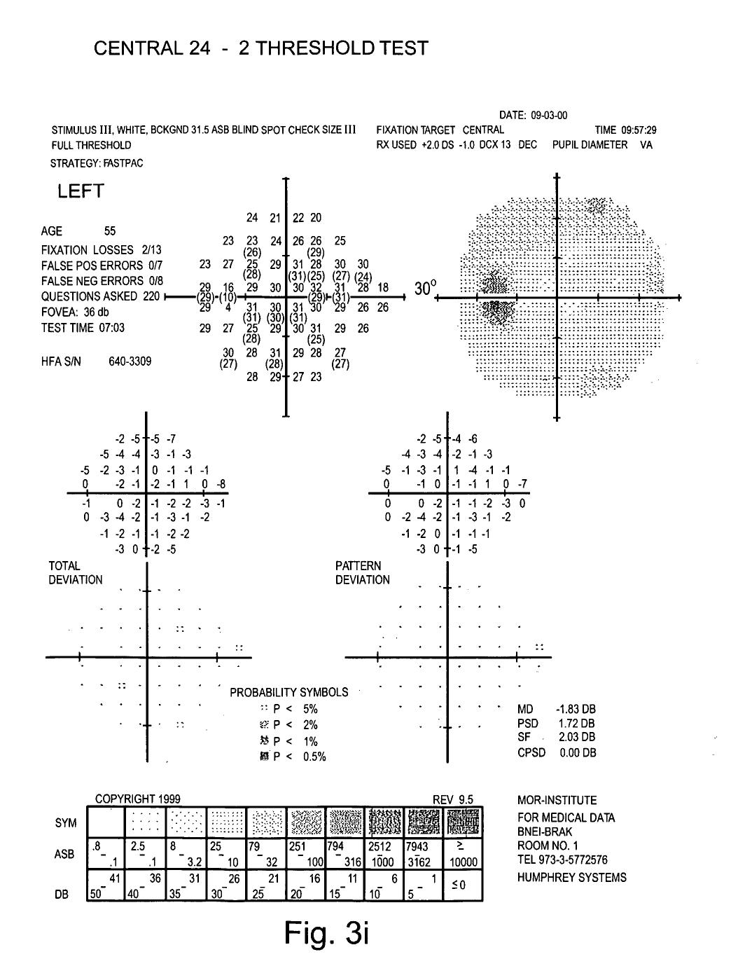

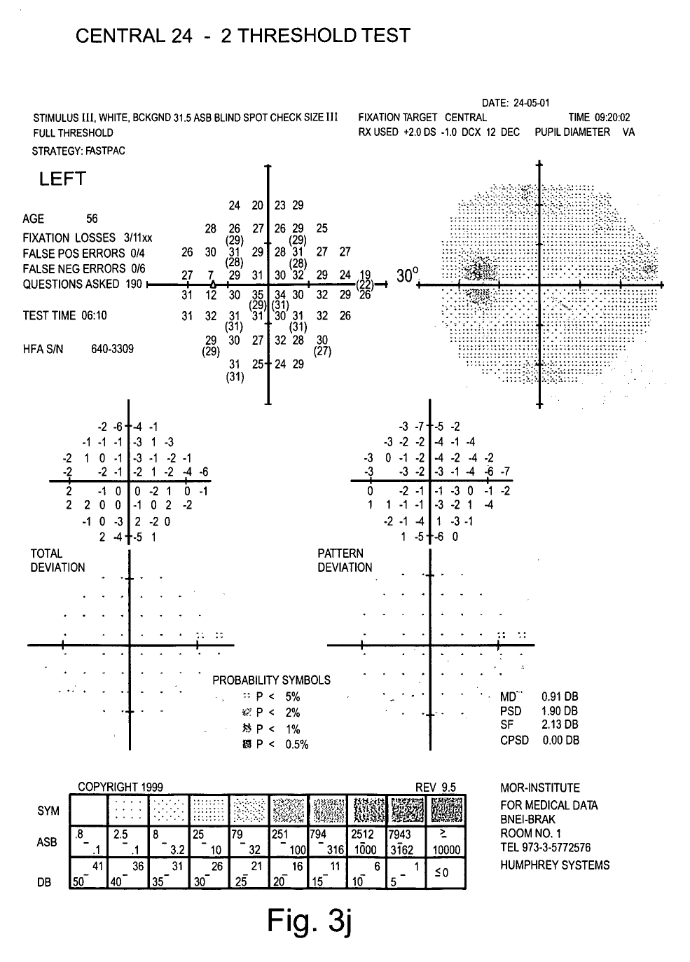

The results from the examination of field of peripheral vision of three patients are illustrated in FIGS. 1A-1C (patient 1); FIGS. 2A-B (patient 2); and FIGS. 3A-J (patient 3). The improvement in each of the patients' visual field testifies to the recovery of the retina and its connections to the brain. This was also seen by examination of the color of the optic nerves in Patient 3, which following treatment were pink in color.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-20. (canceled)

21. A pharmaceutical composition comprising as active ingredients Vitamin C, Vitamin E, Gingko biloba, an amino acid and a physiologically acceptable pharmaceutical carrier or diluent, with the proviso that said amino acid is not glycine.

22. A method of treating a neurodegenerative disorder in a subject, the method comprising providing to a subject in need thereof a pharmaceutical composition comprising as active ingredients Vitamin C, Vitamin E, Gingko biloba, an amino acid and at least one substance selected from the group consisting of a vitamin, a mineral and a herb and a physiologically acceptable pharmaceutical carrier or diluent, thereby treating the neurodegenerative disorder in the subject.

23. The pharmaceutical composition of claim 21, further comprising at least one substance selected from the group consisting of a vitamin, a mineral and a herb.

24. The pharmaceutical composition of claim 23, wherein said vitamin is selected from the group consisting of Vitamin A, Vitamin B1, Vitamin B2, Vitamin B3, Vitamin B5, Vitamin B6, Vitamin B12, Vitamin D, Vitamin H, Choline, Folic acid and Paraamino Benzoic Acid (PABA).

25. The pharmaceutical composition of claim 23, wherein said mineral is selected from the group consisting of Calcium, Chromium, Copper, Iodine, Iron, Magnesium, Manganese, Phosphorus, Potassium, Selenium (Picolinate) and Zinc.

26. The pharmaceutical composition of claim 23, wherein said herb is selected from the group consisting of Allum Sativum (garlic), Black Currant (Ribes nigra), Bromlain, Echinacea, Ginseng (panax), Ginseng (Siberian), Hydrastasis, Medicago sativa (Alfalfa), Passiflora, Ruscus aculeatus, St. John wort (Hypericum perforatum) and Vaccinium myrtillus.

27. The pharmaceutical composition of claim 21, further comprising as least one substance selected from the group consisting of DMAE (Dimethylaminoethanol), Flavonoids (Rutin), Glutathione, Inisitol, Lycopene, Melatonin, Omega 3 fatty acids, Phosphatidyl choline (Lecithine), Phosphatidyl serine, Quercetine and Ubiquitine (Q10).

28. The method of claim 22, wherein the neurodegenerative disorder is selected from the group consisting of myasthenia gravis, Parkinson's disease, multiple sclerosis, amyotrophic lateral sclerosis, diabetic neuropathy, cerebrovascular accident, spinal cord injuries, glaucatomus neuropathy, autoimmune encephalomyelitis, Alzheimer's disease, idiopathic dementia and Huntington's disease.

29. A pharmaceutical composition comprising as active ingredients Vitamin C, Vitamin E, Gingko biloba, Paraamino benzoic acid (PABA), potassium and a physiologically acceptable pharmaceutical carrier or diluent.

30. A pharmaceutical composition comprising as active ingredients Vitamin C, Vitamin E, Gingko biloba, Selenium, Zinc, Vitamin A, Potassium, magnesium and a physiologically acceptable pharmaceutical carrier or diluent.

31. The pharmaceutical composition of claim 30, wherein an amount of said active ingredients is as set forth in Table 2, column 2.

32. A pharmaceutical composition comprising as active ingredients Vitamin C, Vitamin E, Gingko biloba, Selenium, Zinc, Vitamin A, Potassium, magnesium, vitamin B₁, Vitamin B₂, Vitamin B₃, Vitamin B₅, Vitamin B₆, Vitamin B₁₂, Folic acid, PABA and a physiologically acceptable pharmaceutical carrier or diluent.

33. The pharmaceutical composition of claim 32, wherein an amount of said active ingredients is as set forth in Table 2, column 3.

34. A pharmaceutical composition comprising as active ingredients Vitamin C, Vitamin E, Gingko biloba, Selenium, Zinc, Vitamin A, Potassium, magnesium, vitamin B₁, Vitamin B₂, Vitamin B₃, Vitamin B₅, Vitamin B₆, Folic acid, PABA and a physiologically acceptable pharmaceutical carrier or diluent.

35. The pharmaceutical composition of claim 34, wherein an amount of said active ingredients is as set forth in Table 2, column 4.

36. A pharmaceutical composition comprising as active ingredients Vitamin C, Vitamin E, Gingko biloba, potassium and a physiologically acceptable pharmaceutical carrier or diluent.

37. A method of treating glaucatomus neuropathy in a subject, the method comprising administering to a subject in need thereof a pharmaceutical composition comprising as active ingredients Vitamin C, Vitamin E, Gingko biloba, Paraamino benzoic acid (PABA) and potassium, thereby treating the glaucatomus neuropathy in the subject.

38. The pharmaceutical composition of claim 29, further comprising at least one substance selected from the group consisting of Zinc, Selenium picolinate, Vitamin A, Vitamin B₁, Vitamin B₂, Vitamin B₃, Vitamin B₅, Vitamin H, folic acid, Chromium, Manganese, Flavonoid, Phosphatidyl choline, Dimethylaminoethanol, Omega 3 fatty acid, Arginine and Vaccinium myrtillus extract.

39. The pharmaceutical composition of claim 38, wherein the pharmaceutical composition further comprises one or more substances selected from the group consisting of Vitamin B₆, Vitamin B₁₂ Vitamin D, choline, Calcium, Bromlain, Copper, Iodine, Magnesium, Phosphorus, Phosphatidyl serine, Quercetine, Ubiquitine, Glutathione, Inisitol, melatonin, Aspartate, Cysteine, Glutamate, Glutamine, Glycine, Histidine, Isoleucine, Leucine, Lysine, Methionine, Phenylalanine, Proline, Serine, Threonine, Tryptophan, Tyrosine, Valine, Allum Sativum (garlic) Black Currant (Ribes nigra), Echinacea, Ginseng (panax), Ginseng (Siberian), Hydrastasis, Medicago sativa (Alfalfa), Passiflora, Ruscus aculeatus and St. John wort (Hypericum perforatum).

40. The pharmaceutical composition of claim 39, wherein said substance is selected from the group consisting of Magnesium, Vitamin B₆, Vitamin B₁₂ and phosphatidyl serine.

41. The pharmaceutical composition of claim 21, wherein said active ingredients are each in individual unit dosage forms.

42. The pharmaceutical composition of claim 21, wherein the composition is formulated in a unit dosage form.

43. The pharmaceutical composition of claim 41, wherein said unit dosage form is formulated for oral and/or rectal administration.

44. The pharmaceutical composition of claim 42, wherein said unit dosage form is formulated for oral and/or rectal administration.

45. The pharmaceutical composition of claim 41, wherein said unit dosage form is selected from the group consisting of pills, tablets, capsules, gel-capsules and suppositories.

46. The pharmaceutical composition of claim 42, wherein said unit dosage form is formulated for oral and/or rectal administration.

47. The composition of claim 41, wherein said unit dosage form comprises 500-6000 mg of said Vitamin C, 200-8000 IU of said Vitamin E and 40-160 mg of said Gingko biloba extract.

48. The composition of claim 42, wherein said unit dosage form comprises 500-6000 mg of said Vitamin C, 200-8000 IU of said Vitamin E and 40-160 mg of said Gingko biloba extract.
